(19) United States
(12) Patent Application Publication
(10) Pub. No.: US 2016/0100947 A1
(43) Pub. Date: Apr. 14, 2016

(54) DEVICE FOR FIXING A CRANIAL LIMB TO THE CRANIAL CROWN TO BE PLACED IN THE CRANIOTOMIAL HOLE OR CUTTING

(71) Applicant: NTPLAST S.R.L., Rivalta Scriva Tortona (IT)

(72) Inventors: Moreno CARVANI, Rivalta Scrivia Tortona (IT); Igino Romolo GAZZANI, Rivalta Scrivia Tortona (IT); Piero CAVIGLIASSO, Rivalta Scrivia Tortona (IT)

(73) Assignee: NTPLAST S.R.L., Rivalta ScriviaTortona (IT)

(21) Appl. No.: 14/786,495

(22) PCT Filed: Apr. 24, 2013
(86) PCT No.: PCT/IT2013/000122

§ 371 Date: Oct. 22, 2015

(51) Int. Cl. A61F 2/28

(52) U.S. Cl. CPC A61F 2/2875

(57) ABSTRACT

A device is described, for fastening a cranial limb to the cranial crown adapted to be positioned both into a craniotomial hole and into a craniotomial cutting, comprising: a cortical supporting riser; first closing means operatively connected to the riser; second closing means to be fastened to the riser to complete its closing; a handle connected to the cortical riser; and a base for a better cooperation with the elastic arms; the first closing means are composed of at least two elastic arms operatively connected to the cortical riser and that end each one in at least one small transverse arm.

The present invention refers to a device for fixing a cranial limb to the cranial crown to be placed in the craniotomial hole or cutting. An applying device dedicated for such device and a process for using such device are also described.

The present invention is an improvement of the device disclosed in document WO-A1-2009/044421 of the same Applicant.

As known, craniotomy, namely incision and cutting a bone limb of the cranial crown, is the compulsory neuro-surgical procedure for treating any intra-cranial lesion.

The bone limb is cut by performing one or more drill holes, according to two preferred procedures. The first procedure provides for the use of a cutting blade or perforating device applied to the pneumatic drill, that, with free hand, from the key hole detach the dura mater below and simultaneously etches the bone. The second procedure, instead, provides that, with a curved periosteum-detaching device, the dura mater is cut from the bone between a drill hole and another, and, afterwards, using a guide, a saw wire is passes that, pulled upwards at its two ends with wide hands, cuts the bone one segment at a time.

At the end of the neuro-surgical intervention, after having sutured the dura mater and suspended the edges to the bone, the limb is laid again in the opening and fastened with separate metal or wire, staples, made pass through small drill holes coupled on the free edge of the cranial bone.

It is however clear that this type of solution does not allow, in general, an aesthetically acceptable closure, since it is not always able to avoid that the bone limb can be projected, recessed, slanted or rotated.

Object of the present invention, therefore, is solving the above prior art problems, by providing a device for fixing a cranial limb to the cranial crown that allows a correct recovery and attachment of the bone limb to the edge of the cranial crown and simultaneously closes the holes obtained for the craniotomy.

Another object of the invention is providing a device that allows having a lower tension on the brain, even in case a brain edema occurs after the surgery.

With respect to the invention disclosed in the above mentioned patent WO-A1-2009/044421, that necessarily requires the presence of drill holes to be placed, the present invention can be inserted indifferently into the craniotomial hole or into the craniotomial cutting, thereby allowing to fix the limb in many points, even when a single hole has been drilled, or this latter one has an irregular shape. Moreover, an applying device is described, dedicated to simplify the assembly of the device.

Another object of the present invention is making a lower base to be assembled on the above mentioned device to improve its functionality in the following ways:

increasing the resistance to compression loads that could act on the operculum after the craniotomy closure (ex. accidental impacts to which the patient could be subjected), due to a greater abutment surface;

allowing the elastic arms of the device to easily slide during the positioning phase, independently from the presence of irregularities in the internal cranial surface; and

more uniformly distributing the loads onto the internal cranial surface.

As optional feature, the base can be equipped with a centring element from which, when the device is placed inside a hole obtained by the drill, the operculum is better centred in the craniotomy.

These and other objects are obtained by a device for fixing a cranial limb to the cranial crown to be placed into the craniotomial hole or into the craniotomial cutting as described in claim 1.

Further features of the invention are defined in the dependent claims.

It is intended that all claims are an integral part of the present document.

Further objects and advantages of the present invention will be clear from the following description and from the attached drawings, provided merely as a non-limiting example, in which:

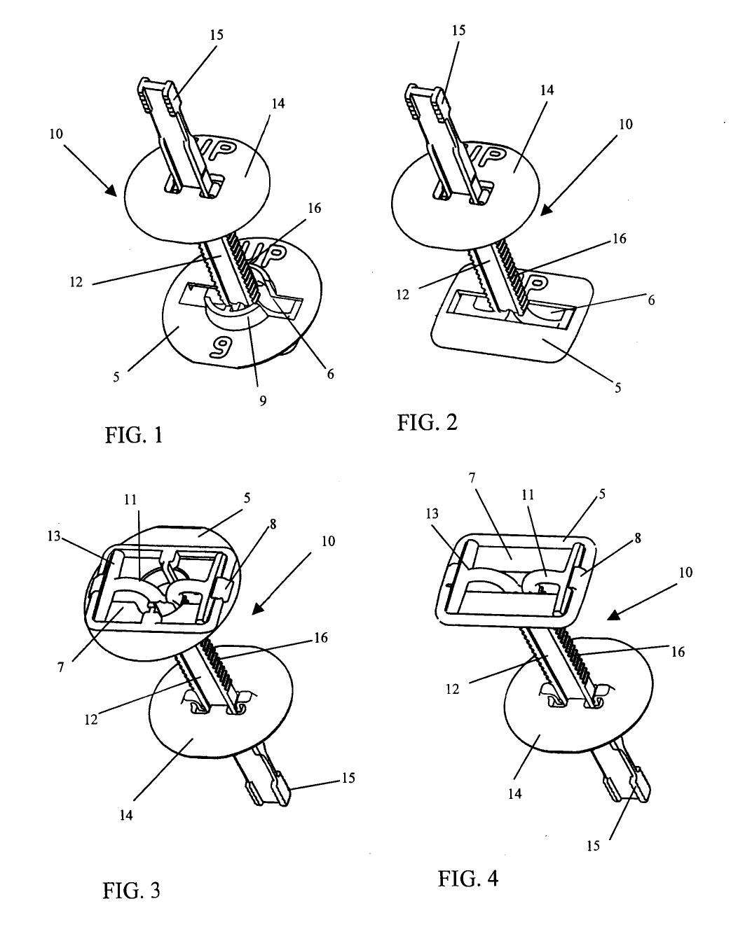

FIG. 1 is a top perspective view of a first preferred embodiment of the device for fixing a cranial limb to the cranial crown to be placed into the craniotomial hole or the craniotomial cutting;

FIG. 2 is a top perspective view of a second preferred embodiment of the device for fixing a cranial limb to the cranial crown to be placed into the craniotomial hole or the craniotomial cutting;

FIG. 3 is a bottom perspective view of the device of FIG. 1;

FIG. 4 is a bottom perspective view of the device of FIG. 2;

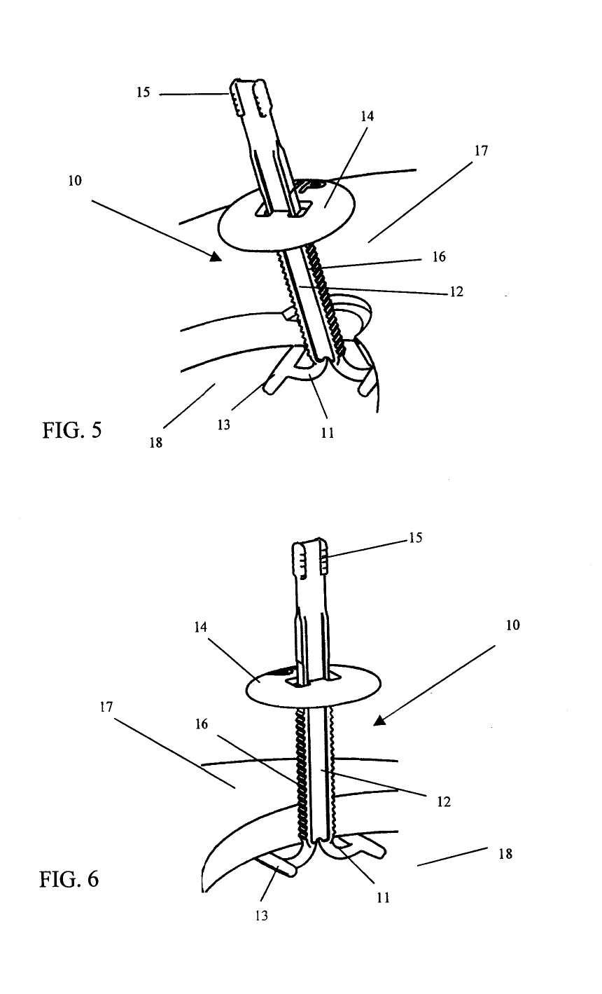

FIG. 5 is a view of the device of FIG. 1 or 2 in a first step of its use in the craniotomial hole;

FIG. 6 is a view of the device of FIG. 1 or 2 in a first step of its use in the craniotomial cutting;

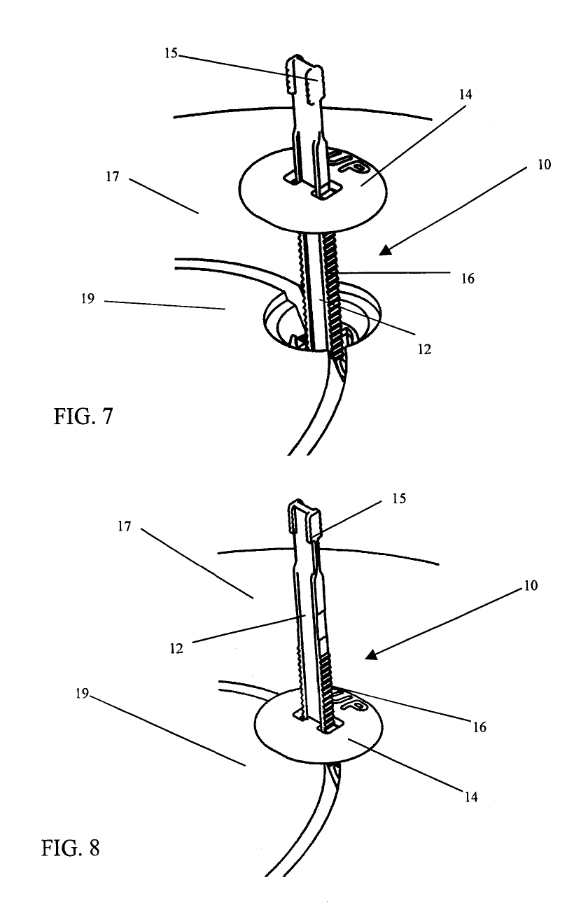

FIG. 7 is a view of the device of FIG. 1 or 2 in a second step of its use in the craniotomial hole;

FIG. 8 is a view of the device of FIG. 1 or 2 in a second step of its use in the craniotomial cutting;

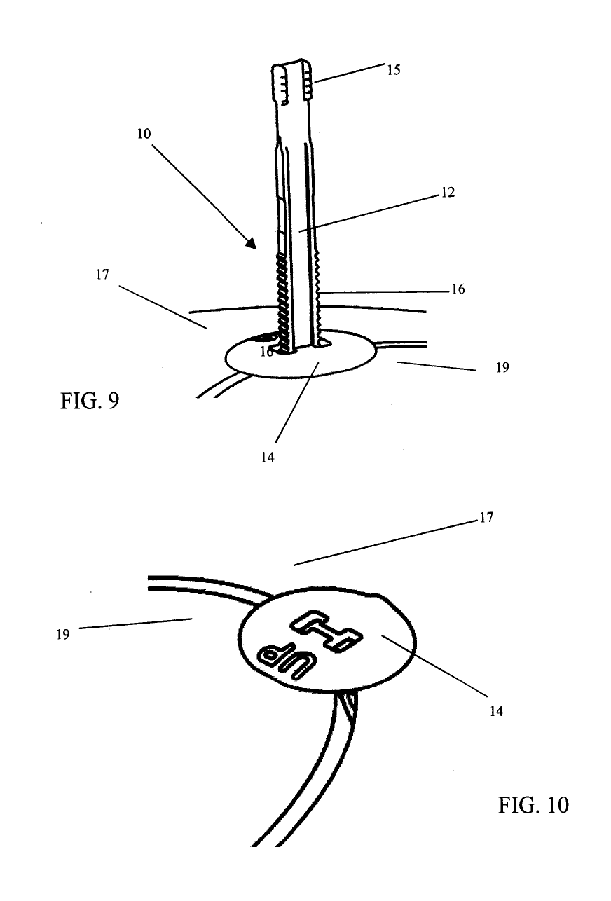

FIGS. 9 and 10 are views of the device of FIG. 1 or 2 in the final steps of its use in the craniotomial hole/cutting;

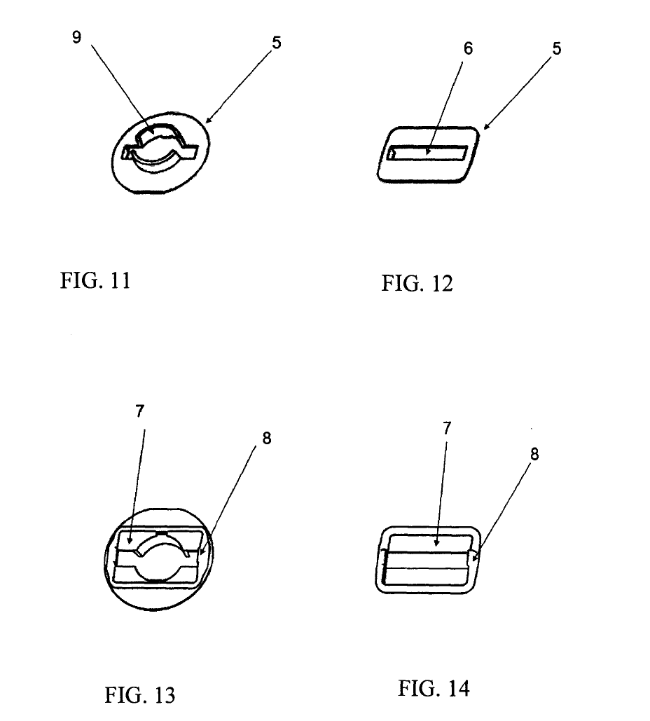

FIGS. 11 and 13 are a top view and a bottom view (this latter one that better points out the blocking system of the small elastic arms of the device of FIG. 1), respectively, of a first embodiment of the base to be used with the device of FIG. 1;

FIGS. 12 and 14 are a top view and a bottom view (this latter one that better points out the blocking system of the small elastic arms of the device of FIG. 1), respectively, of a second embodiment of the base to be used with the device of FIG. 2;

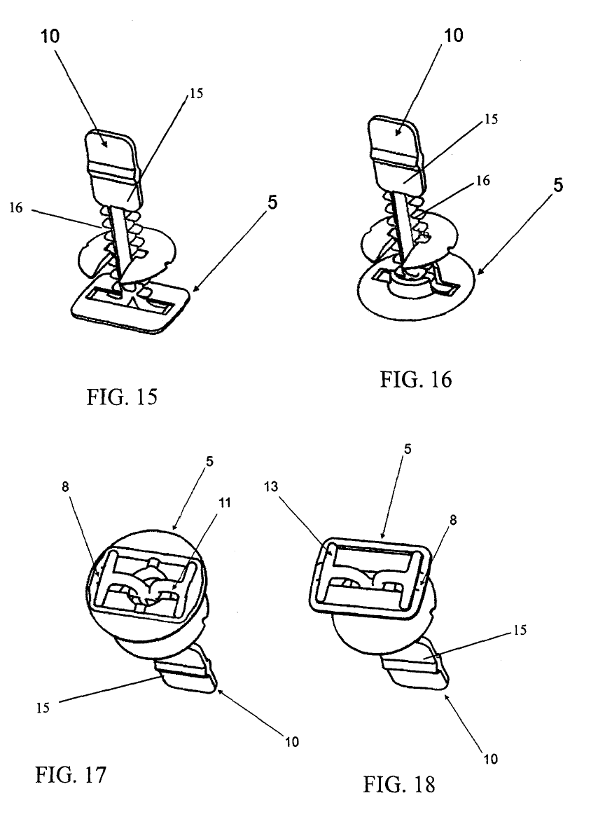

FIGS. 15 and 17 are a top view and a bottom view, respectively, of a third embodiment of the device of the present invention; and

FIGS. 16 and 18 are a top view and a bottom view, respectively, of a fourth embodiment of the of the device of the present invention.

With particular reference to the cited Figures, the device for fixing a cranial limb to the cranial crown to be placed into the craniotomial hole or into the craniotomial cutting, according to the present invention, is globally designated by reference number 10.

The device 10 has elongated elastic arms 11, joined to the cortical riser 12, and ending in small elongated arms 13 placed transversally with respect to the axis of the elongated elastic arms 11, and an upper plate 14, that will have to be inserted into the upper part of the cortical riser 12.

The cortical riser 12 will be equipped with a handle 15 and a plurality of fastening elements 16 (which can be, for example, small teeth as shown in FIGS. 1 to 9, or transverse projections as shown in FIGS. 15 to 18) placed at as many levels along the length of the riser 12, fastening elements 16 that will be used for inserting the upper plate 14.

The cortical riser 12, that connects the elastic arms 11 and the upper plate 14, due to the elasticity of the arms 11, allows a fine adjustment to the bone thickness.

The arrangement of the inventive device 10 therefore provides that the first closing means 11, 13 are connected to the distal end of the cortical riser 12 and the second closing means 14 are adapted to be fastened to the cortical riser 12 in a position next to the distal end of the cortical riser 12, in such a way that, when the device 10 is in its operating fastening position, the first closing means 11, 13 are placed against the internal distal cranial surface and the second closing means 14 are placed against the external near cranial surface.

By examining in more detail the application modes of the cranial fastening device 10 of the invention, it must be noted that the instrument operator prepares the right amount of devices 10 suitable for fixing the craniotomy.

Such operations are performed by choosing the more suitable number of devices 10, depending on the width and shape of the craniotomy.

In order to use the device 10 in practice, an applying device (not shown) is preferably, but not exclusively, provided, adapted to operate with the above described device 10; such applying device comprises a seat adapted to support the upper plate 14 and to allow the operating coupling between such plate 14 and the cortical riser 12 when the craniotomial hole or the craniotomial cutting has been closed.

In order to position the device 10 into the craniotomial hole, the following operations are provided.

A surgeon inserts the device 10 into the craniotomial hole (FIG. 5) with the cortical riser 12 placed at the hole centre and perpendicular to the cranial surface, and so that part of the small cross arms 13 are placed in the space between the cranial crown 17 and the dura mater 18.

The bone operculum 19 is repositioned as shown in FIG. 7. In this way, the cortical riser 12 will project from the craniotomial hole.

Afterwards, the surgeon verifies the correct position of the operculum 19, and stretches, by means of the handle 15, the cortical riser 12 and the elastic arms 11 in order to be able to insert the upper plate 14 next to one of the fastening levels 16, like in FIG. 9. When inserting the upper plate 14, the surgeon uses the above-described applying device. In this way, the craniotomial hole will be closed. Finally, the surgeon removes the handle 15 with a cutting device (FIG. 10).

In order to position the device 10 into the craniotomial cutting, the following operations are provided.

A surgeon inserts the device 10 into the craniotomial cutting (FIG. 6) so that an end of the small transverse arms 13 is placed in the space between the cranial crown 17 and the dura mater 18, with the elastic arms 11 aligned along the direction of the craniotomial cutting and the cortical riser 12 perpendicular to the cranial surface.

The bone operculum 19 is rested onto the other ends of the small transverse arms 13, as shown in FIG. 8. In this way, the riser 12 will project between the cranial crown 17 and the operculum 19.

Afterwards, the surgeon verifies the correct position of the operculum 19, and stretches, by means of the handle 15, the cortical riser 12 and the elastic arms 11 to be able to insert the upper plate 14 next to one of the fastening levels 16, like in FIG. 9. When inserting the upper plate 14, the surgeon uses the above-described applying device 20. Finally, the surgeon removes the handle 15 with a cutting device (FIG. 10).

The device 10 of the invention further comprises, as shown in the various Figures and in detail in FIGS. 11 to 14 in two preferred, but not limiting, configurations thereof, a lower base 5, preferably made of a biocompatible elastic plastic material, to be assembled with the device for fastening a cranial limb and for simultaneously closing the holes obtained for performing the craniotomy.

With particular reference to FIGS. 11 to 14 actually, the lower base according to the present invention is globally designated by numeric reference 5; the device for fastening a cranial limb to the cranial crown and for simultaneously closing the craniotomy holes, as previously described, is globally designated by numeric reference 10.

The lower base 5 has a cut-out 6 through which the device 10 is passes, and a seat 7 where the small transverse arms 13 linked to the elastic arms 11 are housed and slide. In this way, by sliding the small transverse arms 13 when positioning the device 10, independently from the presence of irregularities in the internal cranial surface, a better application functionality of the device 10 is obtained.

The small transverse arms 13 are blocked, preventing them from being excessively and uselessly opened, by the same shape of the seat 7 of the base 5, or by suitable fastening means 8 provided at two ends of the seat 7 itself (as can be seen in FIGS. 3, 4, 17 and 18).

A second version of the lower base 5, shown in FIGS. 1, 11 and 16, has a centring system 9, provided for using the device 10 in holes obtained by the drill.

The cranial fastening device 10 of the invention is suitable for all craniotomy cases, since its shape and its suitability to the bone thickness allow keeping the bone limb tended to the cranial crown edge, both on the internal and on the external margin, in order to allow the correct ossification.

At the same time, the elasticity of the bio-compatible plastic material being used guarantees a lower tension on the brain after an operation, namely when brain edema conditions could occur and therefore a swelling of the brain itself.

From the above description, the features are clear for the device 10 for fastening a cranial limb to the cranial crown to be positioned into the craniotomial hole or the craniotomial cutting, object of the present invention, and its advantages are also clear.

Moreover, in the practice of the invention, materials, shapes, sizes of the shown details could be changed according to needs and they could be replaced by other technically equivalent arrangements.

Finally, it is clear that numerous variations could be made to the device 10 for fastening a cranial limb to the cranial crown to be positioned into the craniotomial hole or the craniotomial cutting, object of the present invention, without thereby departing from the scope of the present invention, as defined by the enclosed claims.

1.-8. (canceled)

9. A device for fastening a cranial limb to a cranial crown adapted to be positioned both into a craniotomial hole and into a craniotomial cutting, the device comprising:
a) at least one cortical supporting riser;
b) a first closing means operatively connected to the cortical riser;
c) a second closing means adapted to be fastened to the cortical riser to complete its closing; and
d) at least one handle removably connected to the cortical riser and adapted to drive the device to take it from its rest position to its operating fastening position; and
wherein the first closing means is connected to a distal end of the cortical riser and the second closing means is adapted to be fastened to the cortical riser in a near position of the distal end of the cortical riser, so that, when the device is in its operating fastening position, the first closing means is placed against the internal distal cranial surface and the second closing means is placed against the external near cranial surface, the first closing means being composed of at least two elongated elastic arms operatively connected to the cortical riser, each one of the elongated elastic arms ending in at least one small elongated arm placed transversally with respect to the elongated elastic arms.

10. The device of claim 9, where the device is further equipped with at least one lower base to be assembled with the device for fastening a cranial limb and for simultaneously closing holes obtained to perform the craniotomy.

11. The device of claim 9, where the lower base has a cut-out through which the device passes, and a seat where the small transverse arms linked to the elastic arms are housed and slide.

12. The device of claim 11, where the small transverse arms are blocked when opening by a shape of the seat of the lower base.

13. The device of claim 11, where the small transverse arms are blocked when opening by fastening means provided at two ends of the seat of the lower base.

14. The device of claim 10, where the lower base further comprises a centering system provided to be used when the device is employed in holes obtained with a drill.

15. The device of claim 9, where the device is composed of elastic material made of bio-compatible plastic.

16. The device of claim 9, where the second closing means further comprises an upper plate adapted to be inserted into the upper part of the cortical riser.

17. The device of claim 16, where the cortical riser further comprises a plurality of fastening elements placed at as many levels along a length of the riser, where the fastening elements are adapted to engage the upper plate after its operating coupling with the riser.
